(19) United States
(12) Patent Application Publication
(10) Pub. No.: US 2002/0078958 A1
(43) Pub. Date: Jun. 27, 2002

(54) Infant CPAP system with airway pressure control

(75) Inventor: Alex Stenzler, Orange, CA (US)

Correspondence Address: LYON & LYON LLP, 633 WEST FIFTH STREET, SUITE 4700, LOS ANGELES, CA, 90071, US

(73) Assignee: SensorMedics Corporation

(21) Appl. No.: 09/746,948

(22) Filed: Dec. 21, 2000

(51) Int. Cl. A62B 7/00; A61M 16/00; F16K 31/02; A62B 9/02; A62B 18/02; A62B 9/06; A61M 15/08

(52) U.S. Cl. 128/204.21; 128/204.18; 128/205.24; 128/205.25; 128/207.14; 128/207.18

(57) ABSTRACT

A device for delivering continuous positive airway pressure (CPAP) to an infant includes a pressurized source of gas containing oxygen, a variable flow control valve, a respiratory breathing circuit including a patient interface device at a terminal portion thereof, at least one pressure sensor disposed in the breathing circuit for measuring the airway pressure of the infant, and a controller for controlling the variable flow control valve. The pressure sensor samples the airway pressure at a high sample rate and reports information to the controller. The controller compares the measured pressure with the set-point pressure and controls the flow through the variable flow control valve to maintain a constant airway pressure through the infant's entire respiratory cycle.

### BACKGROUND OF THE INVENTION

The invention generally relates to devices and methods for delivering continuous positive airway pressure to infants or neonates. More specifically, the present invention relates to a device and method for dynamically controlling the airway pressure during an infant's entire respiratory cycle.

It is critical that when a baby is born, the baby quickly begins to breathe on its own. Unfortunately, newborns and pre-term infants are susceptible to a variety of lung-type ailments which may progress into respiratory distress syndrome. Different factors may contribute to the inability of infants to sustain independent ventilation. For example, pulmonary immaturity and increased chest wall compliance may result in the lungs of the infant being underinflated, i.e., low functional residual capacity (FRC), causing a condition called atelectasis. This loss of lung volume results in alterations in ventilation to perfusion ratios and low blood oxygen levels. For infants suffering from respiratory distress syndrome, continuous positive airway pressure (CPAP) is typically administered. CPAP is the application of positive pressure to the airways of a spontaneously breathing patient throughout the respiratory cycle. CPAP stabilizes the chest wall and increases the mean airway pressure to thereby increase the FRC. CPAP also improves ventilation-perfusion relationships and potentially reduces oxygen requirements.

The delivery of continuous positive airway pressure is accomplished by the use of a positive air flow source that provides oxygen to a patient circuit. The patient circuit typically interfaces with the infant using nasal prongs, nasopharyngeal prongs, an endotracheal tube, a nasopharyngeal tube, head box, or mask. Nasal continuous positive airway pressure (NCPAP) has been shown to be beneficial in increasing oxygenation and decreasing work of breathing in infants.

Current CPAP devices typically depend on controlling a CPAP pressure valve to control pressure within the breathing circuit. A pressure valve is used on the gas leaving the breathing circuit, for example, in CPAP ventilators and CPAP underwater systems. Alternatively, other CPAP devices merely set a set-point pressure and allow the effective airway pressure to fluctuate as the infant breathes through the entire respiratory cycle. In current infant CPAP devices, when CPAP is delivered to the infant during inhalation, the infant must work against the transient decrease in inspiratory pressure to move gas into the lungs. Similarly, during exhalation, the infant must breathe against the transient increase in pressure caused by the incoming flow of gas from the interface device (i.e., nasal prongs, nasopharyngeal prongs, endotracheal tube, nasopharyngeal tube, head box, or mask). This increases the amount of work needed to exhale.

There is a need for device and method for delivering CPAP to an infant that minimizes the amount of work needed for spontaneous breathing. The device preferably controls the amount of air delivered to the patient circuit to maintain a constant airway pressure in the infant. Preferably, the device and method employ a microprocessor-controlled feedback arrangement to dynamically control the airway pressure throughout the entire respiratory cycle.

### SUMMARY OF THE INVENTION

In a first aspect of the invention, a device for delivering continuous positive airway pressure (CPAP) includes a pressurized source of gas containing oxygen and a variable flow control valve in fluidic communication with the pressurized source of gas. The device further includes a respiratory breathing circuit terminating at a patient interface device, wherein the output of the variable flow control valve is in fluidic communication with the respiratory breathing circuit. At least one pressure sensor is located in the breathing circuit at the patient interface device for measuring the airway pressure during the infant's respiratory cycle. The device also includes a controller for controlling the variable flow control valve. The controller receives a signal from at least one pressure sensor. In response to the signal corresponding to the measured airway pressure of the infant, the controller modulates the variable flow control valve to maintain a constant airway pressure during the infant's respiratory cycle.

In a second aspect of the invention, the embodiment of the first invention further includes a humidifier disposed within the respiratory breathing circuit upstream of the patient interface device. In addition, a gas mixing device is connected to the pressurized source of gas to control oxygen concentration. The pressure sensor is a high-speed electronic pressure sensor having a sample rate of at least 100 samples per second.

In a third aspect of the invention, a method is disclosed for delivering constant airway pressure to a spontaneously breathing infant via a patient interface device connected to a respiratory breathing circuit containing pressurized, oxygenated gas. The method includes the steps of repeatedly measuring the airway pressure of the infant at the patient interface device using a high-speed electronic pressure sensor having a sample rate of at least 100 samples per second. Signals corresponding to the measured airway pressure are sent to a controller. The measured airway pressure is compared with a set-point airway pressure. A variable flow control valve disposed upstream of the respiratory breathing circuit is modulated by the controller so as to maintain a constant airway pressure near the set-point airway pressure during the infant's complete respiratory cycle.

It is an object of the invention to provide a device for maintaining constant airway pressure in an infant receiving continuous positive airway pressure therapy. It is a further object of the invention to reduce the amount of energy or work required for infants who receive CPAP therapy. To this end, it is also an object of the invention to control the airway pressure during the infant's respiratory cycle by dynamically increasing gas flow during inhalation and dynamically decreasing gas flow during exhalation.

### BRIEF DESCRIPTION OF THE DRAWINGS

FIG. 1 schematically illustrates a device for delivering CPAP to an infant or neonate according to an embodiment of the invention.

FIG. 2 schematically illustrates a nasal prong including a pressure sensor used to measure the airway pressure of an infant or neonate.

FIG. 3 is a graph illustrating airway pressure as a function of time showing the respiratory cycle of an infant or neonate. The set-point airway pressure is shown together with measured airway pressure for a conventional CPAP device and a CPAP device according to the present invention.

### DETAILED DESCRIPTION OF THE PREFERRED EMBODIMENTS

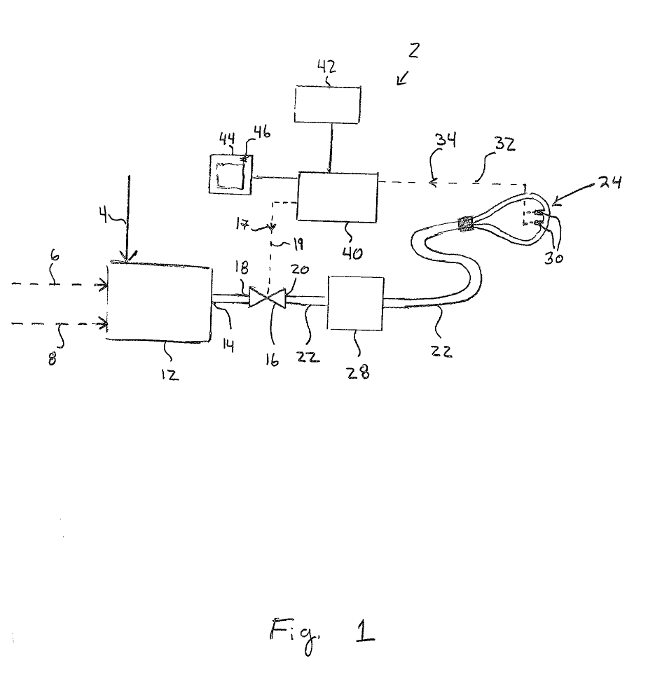

Referring now to FIG. 1, a CPAP device 2 is shown in schematic representation. The device 2 includes a pressurized source of gas 4 that includes at least some portion of oxygen. The pressurized source of gas 4 may originate from a wall supply of air/oxygen, such as that found within hospitals and medical facilities. Alternatively, the pressurized source of gas 4 may originate from a pressurized cylinder or cylinders (not shown). The oxygen may be combined with air, nitrogen, or other gas in a single stream as shown, for example, by element 4 in FIG. 1. An alternative arrangement is to separate the oxygen gas 6 from other gases 8 which might include air and/or nitrogen. This is shown by dashed lines 6, 8 in FIG. 1.

The pressurized source of gas 4 preferably enters into a gas mixer 12. The gas mixer 12 preferably is an air/oxygen blender that provides gas with variable inspired oxygen concentration levels. The oxygen concentration in the gas can be controlled via the mixer 12, or alternatively, via the controller 40 (discussed below). The mixed gas 14 from the gas mixer 12 next passes to an input 18 of the variable flow control valve 16. Preferably, the variable flow control valve 16 is a proportional flow control valve that has a high frequency response and precisely controls, i.e., modulates, the flow of gas output from the variable flow control valve 16. While a proportional flow control valve 16 is preferred, other types of valves 16 may be employed. It is preferable that the variable flow control valve have a fast response time. The variable flow control valve 16 receives a control signal 17 via signal line 19 from controller 40. The control signal 17 will increase or decrease the flow of gas depending on the instructions stored within the controller 40.

The modulated gas passes out of the output 20 of the variable flow control valve 16 to the respiratory breathing circuit 22. The respiratory breathing circuit 22 includes flexible tubing used to transport the respiratory gases to a patient interface device 24. Preferably, a humidifier 28 is disposed within the respiratory breathing circuit 22 upstream of the patient interface device 24. The humidifier 28 alters the water content of the respiratory gases. Preferably, the humidity of the respiratory gases can be controlled via the humidifier 28, or alternatively, via the controller 40 (discussed below).

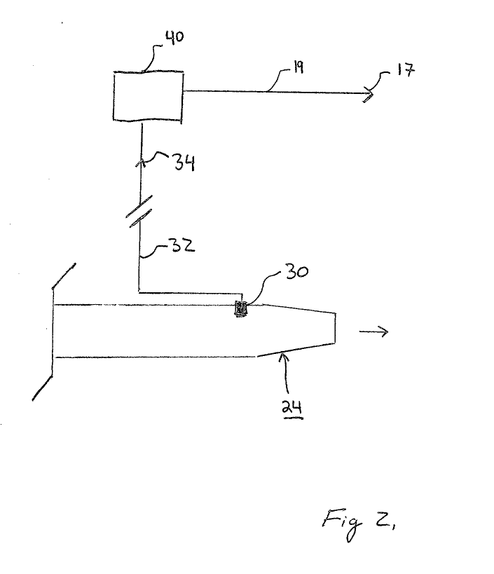

Still referring to FIG. 1, located at one end of the respiratory breathing circuit 22 is the patient interface device 24. The patient interface device 24 can include by way of example, nasal prongs, mask, nasopharyngeal prongs, endotracheal tube, nasopharyngeal tube, and the like. Preferably, the patient interface device 24 comprises nasal prongs (shown in FIGS. 1 and 2).

The patient interface device 24 includes one or more pressure sensors 30. FIGS. 1 and 2 illustrate pressure sensors 30 located in the nasal prongs. (FIG. 2 shows only one nasal prong). Preferably, the pressure sensor 30 is a high-speed electronic pressure transducer that has a sample rate of at least 100 samples per second. Even more preferably, the sample rate of the pressure transducer exceeds 500 samples per second. The high sample rates permit the device 2 to dynamically control the flow and hence airway pressure of the infant. Reference is made throughout this written description to an infant. It should be understood that the term “infant” includes infants, neonates, pre-term infants, and other pediatric patients. The pressure sensor(s) 30 transmit a signal 34 via signal line 32 to the controller 40. The signal line 32 may be external to or integrated with the respiratory breathing circuit 22. As an alternative to the digitally operated pressure sensor 30, an analog-based pressure sensor 30 can also be used provided the pressure sensor 30 has a high response rate.

Referring now to FIG. 2, the end portion of the respiratory breathing circuit 22 preferably includes a jet (as shown in FIG. 2) or venturi design whereby the pressure generated at the patient interface device 24 is controlled by the relative flow of respiratory gases.

Referring to FIGS. 1 and 2, the device 2 includes a controller 40. The controller 40 receives the signal(s) from the pressure sensor(s) 30 and outputs a control signal 17 to the variable flow control valve 16. The controller 40 is preferably a microprocessor-based controller 40 in which instructions may be stored. The instructions or software for the controller 40 may be stored permanently or temporarily therein. The controller 40, in addition to controlling the variable flow control valve 16, may also control other parameters of the device 2 such as oxygen levels of the gas via the gas mixer 12 and humidity levels via the humidifier 28. The controller 40 preferably is coupled to an input device 42 and a display 44. The input device 42 is used to input instructions to the controller 40 such as the parameters of the CPAP gas administration. For example, the input device 42 is used to establish the set-point airway pressure for the infant. The input device 42 might also be used to input safety parameters to the controller 40. For instance, an operator might want an alarm 46 to trigger if the measured pressure falls above or below certain levels. Preferably, the controller 40 reports various parameters to a display 44. The display 44 preferably shows various parameters such as measured airway pressure, set-point airway pressure, oxygen concentration, humidity level, and the like. The controller 40 can also be integrated with other sensors such as a heart rate monitor, pulse oximeter, and transcutaneous CO₂ monitors. The data from these sensors can be displayed on the display 44.

The controller 40 can employ any number of control modes to control the airway pressure of the infant. For example, the controller 40 can be a Proportional (P) controller 40, a Proportional-Integral (PI) controller 40, a Proportional-Integral-Derivative (PID) controller 40, and the like. Preferably, the controller 40 is a microprocessor-based controller 40 in which pressure measurements in the form as electrical signals (analog or digital) are applied as inputs to the controller 40. The controller 40 calculates the output value to drive the variable flow control valve 16. If the control system is analog based, the necessary analog-to-digital and digital-to-analog converters can be incorporated into the controller 40.

With reference to FIGS. 1 through 3, a description of the use and operation of device 2 will now be described. The operator of the device 2, typically a doctor, nurse, or other trained professional, sets a desired set-point airway pressure using the input device 42. The operator attaches the respiratory breathing circuit 22 to the infant using the patient interface device 24. The operator also powers up the device 2 and secures the necessary tubing for the source of pressurized gas 4. The device 2 initially produces a positive airway pressure at the established set-point. Using the feedback arrangement of the pressure sensor(s) 30, controller 40, and variable flow control valve 16, the flow through the variable flow control valve 16 is adjusted to set the flow of respiratory gases through the respiratory breathing circuit 22 to produce the desired set-point airway pressure.

As the infant begins to inhale, there is a transient pressure drop in the airway pressure. The pressure sensor(s) 30 which measure the airway pressure report this pressure drop information to the controller 40. The controller 40 then compares the measured airway pressure with the set-point airway pressure. Since the measured airway pressure is less than the set-point airway pressure, the controller 40 sends a control signal 17 to the variable flow control valve 16 to increase the flow of respiratory gases to compensate for the pressure drop. The airway pressure measurements and reporting to the controller 40 are repeatedly made during the respiratory cycle. During exhalation by the infant, the measured airway pressure begins to rise. The pressure information is reported to the controller 40. Since the measured airway pressure is higher than the set-point airway pressure, the controller 40 sends a control signal 17 to the variable flow control valve 16 to decrease the flow of respiratory gases to compensate for the pressure increase. By rapidly sampling the airway pressure measurements to the controller 40 and modulating the flow of respiratory gases in the respiratory breathing circuit 22, the device 2 is capable of maintaining a substantially constant airway pressure in the infant.

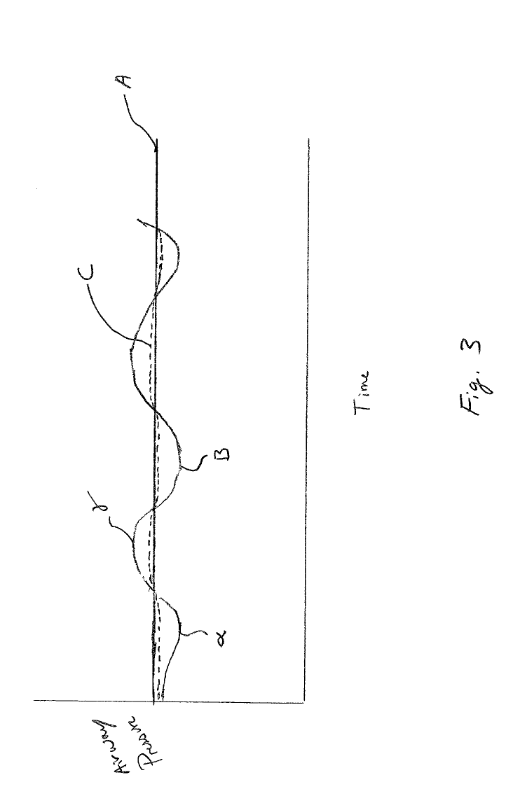

FIG. 3 graphically illustrates the operation of the present device 2. The set-point airway pressure is labeled A. As seen in FIG. 3, the set-point airway pressure is positive and constant. The solid line labeled B illustrates the operation of prior art CPAP infant devices. In these devices, when the infant begins to inhale, a transitory pressure decrease occurs, as shown in FIG. 3 by the portion of solid line B labeled α. Similarly, when the infant begins to exhale, a transitory pressure increase occurs as shown in FIG. 3 by the portion of solid line B labeled γ.

The device 2 according to the present invention, however, substantially reduces or eliminates entirely the transitory pressure increases/decreases in the airway pressure. As stated in more detail above, the device 2 utilizes a feedback arrangement or loop with the pressure sensor(s) 30, controller 40, and variable flow control valve 16 to modulate the flow within the respiratory breathing circuit 22 to maintain a constant airway pressure. The hashed line C in FIG. 3 graphically illustrates the operation of the present device 2. As can be seen, the transitory pressure increases/decreases during the infant's respiratory cycle are substantially reduced or eliminated entirely. It should be understood that hashed line C in FIG. 3 graphically represents the operation of the device 2. While some variation of the airway pressure above and below the set-point airway pressure is seen in FIG. 3 in hashed line C, this variation is shown for illustration purposes. It is preferable that the device 2 operate to minimize any fluctuation of airway pressure above and below the set-point valve.

While embodiments of the present invention have been shown and described, various modifications may be made without departing from the scope of the invention. For example, the controller 40 might be a stand-alone component of the device 2, or it might be integrated with other control electronics. Alternatively, the controller 40 can be implemented with a separate discrete microprocessor or even a separate computer. The invention, therefore, should not be limited, except to the following claims and their equivalents.

What is claimed is:

1. A device for delivering continuous positive airway pressure (CPAP) to an infant comprising:
a pressurized source of gas containing oxygen;
a variable flow control valve, said variable flow control valve including an input and an output, the input being connected to said pressurized source of gas;
a respiratory breathing circuit terminating at a patient interface device, the output of said variable flow control valve connecting with said respiratory breathing circuit;
at least one pressure sensor located in the respiratory breathing circuit at the patient interface device, the at least one pressure sensor measuring the airway pressure of the infant; and
a controller for controlling the variable flow control valve, said controller receiving a signal from the at least one pressure sensor corresponding to the measured airway pressure of the infant, wherein said variable flow control valve is modulated to maintain a constant airway pressure during the infant's complete respiratory cycle.

2. A device according to claim 1, wherein the patient interface device comprises nasal prongs.

3. A device according to claim 1, wherein the patient interface device comprises a mask.

4. A device according to claim 1, wherein the patient interface device comprises nasopharyngeal prongs.

5. A device according to claim 1, wherein the patient interface device comprises an endotracheal tube.

6. A device according to claim 1, wherein the patient interface device comprises a nasopharyngeal tube.

7. A device according to claim 1, wherein the at least one pressure sensor comprises a high-speed electronic pressure transducer having a sample rate of at least 100 samples per second.

8. A device according to claim 1, wherein the at least one pressure sensor comprises a high-speed electronic pressure transducer having a sample rate of at least 500 samples per second.

9. A device according to claim 1, wherein the variable flow control valve comprises a proportional flow control valve.

10. A device according to claim 1, further comprising an input device for setting a set-point pressure to be delivered to the infant.

11. A device according to claim 1 further comprising a display.

12. A device according to claim 1, further comprising a gas mixing device disposed downstream of said pressurized source of gas and upstream of said variable flow control valve.

13. A device according to claim 1, further comprising a humidifier disposed in the respiratory breathing circuit upstream of the patient interface device.

14. A device for delivering continuous positive airway pressure (CPAP) to an infant comprising:
a pressurized source of gas containing oxygen;
a gas mixing device connected to said pressurized source of gas;
a variable flow control valve, said variable flow control valve including an input connected to an output of said gas mixing device;
a respiratory breathing circuit terminating at a patient interface device, the output of said variable flow control valve connecting with said respiratory breathing circuit;
a humidifier disposed within said respiratory breathing circuit upstream of the patient interface device;
at least one high-speed electronic pressure sensor having a sample rate of at least 100 samples per second located in the breathing circuit at the patient interface device, wherein the at least one high-speed electronic pressure sensor measures the airway pressure during the infant's respiratory cycle; and
a controller for controlling the variable flow control valve, said controller receiving a signal from the at least one high-speed electronic pressure sensor corresponding to the measured airway pressure of the infant, wherein said variable flow control valve is modulated to maintain a constant airway pressure during the infant's complete respiratory cycle.

15. A device according to claim 14, wherein the patient interface device comprises nasal prongs.

16. A device according to claim 14, wherein the patient interface device comprises a mask.

17. A device according to claim 14, wherein the patient interface device comprises nasopharyngeal prongs.

18. A device according to claim 14, wherein the patient interface device comprises an endotracheal tube.

19. A device according to claim 14, wherein the patient interface device comprises a nasopharyngeal tube.

20. A device according to claim 14, wherein the at least one pressure sensor comprises a high-speed electronic pressure transducer having a sample rate of at least 500 samples per second.

21. A device according to claim 14, wherein the variable flow control valve comprises a proportional flow control valve.

22. A device according to claim 14, further comprising an input device for setting a set-point pressure to be delivered to the infant.

23. A device according to claim 14 further comprising a display.

24. A method of delivering constant airway pressure to a spontaneously breathing infant via a patient interface device connected to a respiratory breathing circuit containing pressurized, oxygenated gas comprising the steps of:
repeatedly measuring the airway pressure of the infant at the patient interface device using a high-speed electronic pressure sensor having a sample rate of at least 500 samples per second;
reporting signals corresponding to the measured airway pressure to a controller;
comparing the measured airway pressure with a set-point airway pressure; and
modulating a variable flow control valve disposed upstream of the respiratory breathing circuit so as to maintain a constant airway pressure near the set-point airway pressure during the infant's complete respiratory cycle.
